(19) United States
(12) Patent Application Publication
(10) Pub. No.: US 2021/0388299 A1
(43) Pub. Date: Dec. 16, 2021

(54) MANIPULATION APPARATUS AND MANIPULATION METHOD

(71) Applicants: PMT CORPORATION, Kasuya-gun (JP); KYUSHU UNIVERSITY, NATIONAL UNIVERSITY CORPORATION, Fukuoka-shi (JP)

(72) Inventors: Fumihiro SASSA, Fukuoka-shi (JP); Osamu TAKEI, Kasuya-gun (JP); Hajime MOGI, Kasuya-gun (JP)

(21) Appl. No.: 17/344,735

(22) Filed: Jun. 10, 2021

(30) Foreign Application Priority Data
Jun. 12, 2020 (JP) 2020-102019

(51) Int. Cl. C12M 1/32; C12M 1/34; C12M 3/06

(52) U.S. Cl. CPC C12M 23/12; C12M 23/16; C12M 33/06; C12M 41/36

(57) ABSTRACT

A manipulation apparatus and a manipulation method for performing culturing, manipulation, and analysis of minute samples such as cells and microbes on a large scale, the manipulation apparatus including a stage unit on which a predetermined cell incubator is mounted and a probe array unit having a plurality of probes. The probe array unit includes a first probe array and a second probe array. The first probe array includes a plurality of probes arranged side by side at a predetermined pitch along the X-axis direction. The second probe array includes a plurality of probes arranged side by side at a predetermined pitch along the Y-axis direction.

### TECHNICAL FIELD

The present invention relates to a manipulation apparatus and a manipulation method and, more particularly, to a manipulation apparatus and a manipulation method which can efficiently perform culturing, manipulation, and analysis of minute samples such as cells and microbes on a large scale and can perform experiment manipulation using a small number of reagents, etc.

### BACKGROUND ART

In biological experiments, manipulation experiments are performed, accompanying contact manipulation with respect to minute samples, a small number of reagents, etc., such as cell culturing, reagent addition, microbe isolation, and chemical analysis.

Such manipulation experiments are associated with a wide variety of fields such as infectious disease testing, medical examination, regenerative medicine, DNA analysis, drug discovery, and searches for useful microorganisms.

Experiments accompanying such contact manipulation with respect to minute samples such as cells have been performed manually by experimenters or by automated operations via robot systems.

In this case, as a manipulation apparatus that performs contact manipulation for cells, etc., a colony picking apparatus has been used, which brings a needle having a minute distal end into contact with each cell cultivated on a substrate such as a plate (see, for example, Patent Literature 1).

Recently, in biological fields, microarray type analysis systems that can acquire an enormous amount of data have been increasingly used. For example, new academic fields have been developed, including bioinformatics and metagenomic analysis using high-density array devices such as DNA arrays and next-generation DNA sequencers.

A technique for collectively acquiring a large amount of information from a sample such as a cell by combining an array device and a high-sensitivity CCD camera implements the acquisition of 108-bit order information.

Various types of analysis based on such an enormous amount of information are indispensable for the advancement of science and technology, not only for the advancement of current life sciences but also due to the increasing needs for simultaneous multi-parallel analysis/reaction in industrial and chemical fields.

### CITATION LIST

### Patent Literature

[Patent Literature 1] Japanese Published Unexamined Patent Application No. 2011-50259

### SUMMARY OF INVENTION

### Technical Problem

However, while being able to acquire a large amount of information in an analysis operation in a microarray type analysis system, an individual manipulation means for an array element (an immobilized spot, solution well, culturing chamber, etc.) in a cell sample provided for analysis is limited to manual manipulation by an experimenter or manipulation using a robot system including a colony picking device disclosed in Patent Literature 1.

In particular, in performing manipulation requiring contact with samples, such as picking up cell samples, sample splitting, inoculating, mixing, and adding a small number of reagents, the number of manipulations per hour is limited to several hundreds to several thousands.

That is, there is a large discrepancy between the amount of manipulation in operations accompanying contact with cell samples and the amount of information obtained by a microarray type analysis system that can perform several hundred thousand order analysis per manipulation. This has been a factor that significantly limits the large-scale dramatic speed-up of biological experiments.

There are demands for increases in the manipulation efficiency of operations accompanying contact with samples including not only microarray type cell samples but also cell samples cultivated on a plate on which cellular sections are placed or a gel plate formed on a laboratory dish.

The present invention has been made in view of the above points and an object thereof is to provide a manipulation apparatus and a manipulation method which can efficiently perform culturing, manipulation, and analysis of minute samples such as cells and microbes on a large scale and can perform experiment manipulation using a small number of reagents, etc.

### Solution to Problem

In order to achieve the above object, a manipulation apparatus according to the present invention includes a substrate mounting unit on which a predetermined substrate is mounted and a probe array unit that is driven so as to be able to come into contact with a target sample on the predetermined substrate, has a plurality of probes arranged side by side, is provided to face the substrate mounting unit, and is configured to be movable relative to the substrate mounting unit.

In this case, the probe array unit is driven so as to be able to come into contact with a target sample on a predetermined substrate and has a plurality of probes arranged side by side. This allows the probe array unit to efficiently perform manipulation accompanying contact with the target sample via the plurality of probes. Note that the target sample in this case is not limited to a biological sample such as a cell or microbe and includes a reagent, detergent, and disinfectant, etc.

The probe array unit is provided to face the substrate mounting unit and is configured to be able to move relative to the substrate mounting unit. This makes it possible to change the positions of the probe array unit and the substrate mounting unit while the probe array unit faces the substrate mounting unit. This allows each probe to come into contact with a desired position on the substrate.

Arranging a plurality of probes side by side makes it possible to reduce the moving distance of the probe array unit relative to the substrate mounting unit when bringing each of the plurality of probes into contact with a desired position on the substrate.

When the probe array unit includes at least a first probe array having a plurality of probes arranged side by side in the first direction and a second probe array having a plurality of probes arranged side by side in the second direction different from the first direction, it is possible to further efficiently perform manipulation accompanying contact with a target sample via the two probe arrays arrayed in the different directions. Note that in this case, the number of probe arrays is not limited to two, and it is possible to adopt an arrangement including three or more probe arrays.

The probe array unit is configured to be movable back and forth relative to the substrate mounting unit in the X-axis direction and the Y-axis direction perpendicular to the X-axis direction. When the first direction is either the X-axis direction or the Y-axis direction and the second direction is either the X-axis direction or the Y-axis direction and perpendicular to the arraying direction of the first probe array, manipulation accompanying contact with a target sample can be performed more efficiently via the two probe arrays having the probes arrayed in the X-axis direction and the Y-axis direction.

For example, a substrate is segmented into squares in the X-axis direction and the Y-axis direction, and the first probe array or the second probe array is made to continuously act on a plurality of individual squares. This makes it possible to quickly perform manipulation accompanying contact with a target sample. Using two-axis probe arrays including the first probe array and the second probe array makes it possible to efficiently perform manipulation such as picking up cell samples, sample splitting, inoculating, mixing, and adding a small number of reagents along the X-axis direction and the Y-axis direction of the substrate by, for example, making the first probe array in charge of contact manipulation along the X-axis direction of a substrate and the second probe array in charge of contact manipulation along the Y-axis direction of the substrate.

Note that segmentation on squares in this case may include, for example, not only segmentation on a microwell array on which a plurality of independent wells are formed but also segmentation at virtual coordinate positions on a medium, etc., without clear segmentation on a substrate.

When the probe array unit is configured to move back and forth relative to the substrate mounting unit in the X-axis direction and the Y-axis direction perpendicular to the X-axis direction and pivot relative to the substrate mounting unit in the □ direction around the Z-axis perpendicular to the X-axis and the Y-axis perpendicular to the X-axis, and includes a probe array having a plurality of probes arranged side by side in either the X-axis direction or the Y-axis direction, it is possible to more efficiently perform back-and-forth linear movement and rotational movement and manipulation accompanying contact with a target sample via the probe array arranged side by side in one-axis direction.

That is, it is possible to quickly perform manipulation accompanying contact with a target sample by, for example, segmenting a substrate into squares in the X-axis direction and the Y-axis direction and making the probe array continuously act on a plurality of individual squares. In addition, it is possible to efficiently perform manipulation such as picking up cell samples, sample splitting, inoculating, mixing, and adding a small number of reagents along the X-axis direction and the Y-axis direction of the substrate by, for example, making the probe array perform contact manipulation along the X-axis direction of a substrate and making the probe array perform contact manipulation along the Y-axis direction of the substrate upon rotating the probe array unit or the substrate mounting unit by 90°.

Note that segmentation on squares in this case may include, for example, not only segmentation on a microwell array on which a plurality of independent wells are formed but also segmentation at virtual coordinate positions on a medium, etc., without clear segmentation on a substrate.

When the first probe array and the second probe array are arranged substantially in the form of the letter “L,” it is possible to more efficiently perform manipulation accompanying contact with a target sample. That is, the first probe array and the second probe array are arranged as close to each other as possible without causing the respective probe arrays to interfere with each other. This makes it possible to reduce the moving distance by which the probe array unit and the substrate mounting unit are relatively moved when making probes act at a plurality of desired positions on the substrate.

When there is provided a probe processing unit that is formed substantially parallel to the arraying direction of the probes at a position and at a length that allows contact by a plurality of probes and configured to clean and sterilize the probes or add a reagent to the probes, the probe processing unit can collectively clean or sterilize a plurality of probes.

When there is provided a probe contact portion that is configured to be movable substantially parallel to the arraying direction of the probes and comes into contact with a plurality of probes to clean or sterilize the probes or make a target sample adhere to the probes, the probe contact portion can collectively clean or sterilize a plurality of probes or make a target sample adhere to the probes.

When a plurality of probes are formed at a pitch of 100 nm or more to 5 mm or less, it is possible to satisfactorily perform manipulation accompanying contact and handling a target sample such as a cell or microbe.

When the predetermined substrate is a microwell array on which 100 or more wells per cm2 are formed, it is possible to more efficiently perform manipulation accompanying contact with a target sample with respect to a large number of wells on the microwell array. In addition, it is possible to perform, on one microwell array, a series of medium analysis and manipulation such as culturing cells inoculated on some wells of the microwell array, analyzing the cultivated cells, and re-inoculating and culturing the cells on other wells.

In order to achieve the above object, a manipulation method according to the present invention includes a moving step of relatively moving a probe array unit having a plurality of probes provided to face a substrate mounting unit on which a predetermined substrate is mounted and arranged side by side and a contact step of driving the probe at a desired position to bring the probe into contact with a target sample on the predetermined substrate.

In this case, manipulation accompanying contact with a target sample can be efficiently performed via a plurality of probes by performing the moving step of relatively moving the probe array unit having the plurality of probes arranged side by side and the contact step of driving the probe at the desired position to bring the probe into contact with the target sample on the predetermined substrate. Note that the target sample in this case is not limited to a biological sample such as a cell or microbe and includes a reagent, detergent, and disinfectant, etc.

In the moving step, relatively moving the probe array unit having the plurality of probes provided to face the substrate mounting unit and arranged side by side can change the positions of the probe array unit and the substrate mounting unit while the probe array unit faces the substrate mounting unit. This can bring the probe into contact with a desired position on the substrate.

Making the probe array unit have a plurality of probes arranged side by side can reduce the moving distance of the probe array unit relative to the substrate mounting unit when bringing each of a plurality of probes into contact with a desired position on the substrate.

When the probe array unit includes at least a first probe array having a plurality of probes arranged side by side in a first direction and a second probe array having a plurality of probes arranged side by side in a second direction different from the first direction, manipulation accompanying contact with a target sample can be more efficiently performed via the two probe arrays having different arraying directions. Note that the number of probe arrays in this case is not limited to two and the probe array unit including three or more probe arrays can be adopted.

When the first direction is either the X-axis direction or the Y-axis direction and the second direction is either the X-axis direction or the Y-axis direction and perpendicular to the arraying direction of the first probe array, and in addition, in the moving step, the probe array unit is moved back and forth relative to the substrate mounting unit in the X-axis direction and the Y-axis direction, manipulation accompanying contact with a target sample can be performed more efficiently via the two probe arrays having the probes arrayed in the X-axis direction and the Y-axis direction, respectively.

That is, for example, a substrate is segmented into squares in the X-axis direction and the Y-axis direction, and the first probe array or the second probe array is made to continuously act on a plurality of individual squares. This makes it possible to quickly perform manipulation accompanying contact with a target sample. Using two-axis probe arrays including the first probe array and the second probe array makes it possible to efficiently perform manipulation such as picking up cell samples, sample splitting, inoculating, mixing, and adding a small number of reagents along the X-axis direction and the Y-axis direction of the substrate by, for example, making the first probe array in charge of contact manipulation along the X-axis direction of a substrate and the second probe array in charge of contact manipulation along the Y-axis direction of the substrate.

Note that segmentation on squares in this case may include, for example, not only segmentation on a microwell array on which a plurality of independent wells are formed but also segmentation at virtual coordinate positions on a medium, etc., without clear segmentation on a substrate.

When the probe array unit has a probe array having a plurality of probes arranged side by side in the X-axis direction or the Y-axis direction perpendicular to the X-axis, and in addition, in the moving step, the probe array unit is moved back and forth relative to the substrate mounting unit in the X-axis direction and the Y-axis direction and is made to pivot relative to the substrate mounting unit in the □ direction around the Z-axis perpendicular to the X-axis and the Y-axis, it is possible to more efficiently perform back-and-forth linear movement and rotational movement and manipulation accompanying contact with a target sample via the probe arrays arranged side by side in one-axis direction.

That is, it is possible to quickly perform manipulation accompanying contact with a target sample by, for example, segmenting a substrate into squares in the X-axis direction and the Y-axis direction and making the probe array continuously act on a plurality of individual squares. In addition, it is possible to efficiently perform manipulation such as picking up cell samples, sample splitting, inoculating, mixing, and adding a small number of reagents along the X-axis direction and the Y-axis direction of the substrate by, for example, making the probe array perform contact manipulation along the X-axis direction of a substrate and making the probe array perform contact manipulation along the Y-axis direction of the substrate upon rotating the probe array unit or the substrate mounting unit by 90°.

Note that segmentation on squares in this case may include, for example, not only segmentation on a microwell array on which a plurality of independent wells are formed but also segmentation at virtual coordinate positions on a medium, etc., without clear segmentation on a substrate.

This method also includes a culturing step of culturing a target sample on a predetermined substrate and an analysis step of performing image analysis with respect to the target sample cultivated on the predetermined substrate. Repeatedly performing the culturing step and the analysis step with respect to the predetermined substrate can efficiently perform culturing and analysis with respect to cells, etc., on one substrate.

### Effects of Invention

The manipulation apparatus and the manipulation method according to the present invention can efficiently perform culturing, manipulation, and analysis of minute samples such as cells and microbes on a large scale and can perform experiment manipulation using a small number of reagents, etc.

### BRIEF DESCRIPTION OF THE DRAWINGS

FIG. 1A is a schematic view of a manipulation apparatus as the first embodiment of the present invention, and FIG. 1B is a schematic sectional view showing the operation of contact manipulation with a probe;

FIG. 2A is a schematic view of a manipulation apparatus having a cleaning tank and a sterilizing tank, and FIG. 2B is a schematic view of a manipulation apparatus having a mechanical cleaning unit;

FIGS. 3A-F are schematic views showing a sequence of seeding a cell sample cultivated in a specific well of a microwell array into a plurality of wells of the same microwell array in an arbitrary pattern;

FIG. 4 is a schematic view schematically showing how analysis manipulation and culturing manipulation are repeated with respect to a target sample by using the manipulation apparatus;

FIGS. 5A-C are schematic views schematically showing a sequence of performing experiment manipulation between a gel plate and a microwell array by using the manipulation apparatus; and

FIGS. 6A-D are schematic views showing a sequence of seeding a cell sample cultivated in a specific well of a microwell array into a plurality of wells of the same microwell array in an arbitrary pattern.

### DESCRIPTION OF EMBODIMENTS

The embodiments of the present invention will be described with reference to the accompanying drawings for the purpose of understanding the present invention.

### First Embodiment

The first embodiment of the present invention will be described as an example of a manipulation apparatus to which the present invention is applied. The following contents are merely an example of a structure to which the present invention is applied, and the embodiments of the present invention are not limited to the following structure.

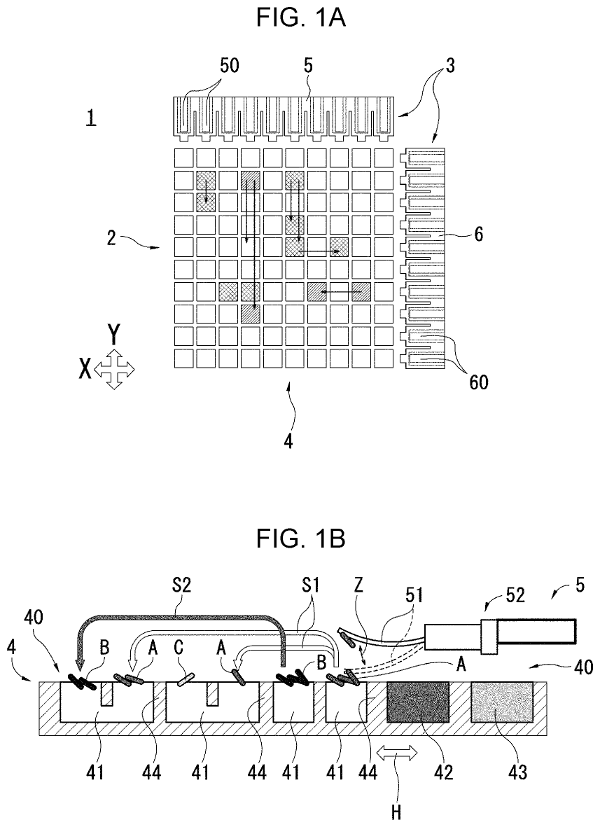

As shown in FIG. 1A, a manipulation apparatus 1 as an example of a manipulation apparatus to which the present invention is applied includes a stage unit 2 on which a predetermined cell incubator is mounted and a probe array unit 3 having a plurality of probes. Note that the predetermined cell incubator in this case corresponds to a predetermined substrate in the claims of the present application. In addition, the stage unit 2 in this case corresponds to a substrate mounting unit in the claims of the present application. Furthermore, the probe array unit 3 in this case corresponds to a probe array unit in the claims of the present application.

In the following description, referring to FIG. 1A, the left-right direction of the drawing surface will be referred to as the X-axis direction, and the up-down direction of the drawing surface will be referred to as the Y-axis direction. Referring to FIG. 1A, the left, right, up, and down of the drawing surface will be respectively referred to as the left side, right side, upper side, and lower side. In addition, with reference to FIG. 1B, the microwell array 4 side viewed from a probe 5 will be referred to as the vertical downside, and the probe 5 side viewed from a microwell array 4 will be referred to as the vertical upside. In addition, with reference to FIG. 1B, the direction connecting the vertical upside and the vertical downside will be referred to as the Z-axis direction or vertical direction.

The cell incubator is a substrate that allows cell culturing (including a cell sample or microbe sample), and is, for example, a microwell array on which a plurality of individual wells are formed, a laboratory dish, and a slide on which sliced segments of a cell are placed, etc. In this case, the microwell array 4 is exemplified as a cell incubator (see FIGS. 1A and 1B).

A plurality of concave wells 40 of the microwell array 4 are filled with a gel culture medium 41, a reagent gel 42, and a disinfectant gel 43. The wells 40 are partitioned by partition walls 44 so as to prevent cross-contamination among the cells, etc., inoculated in the respective gel culture media 41 (see FIG. 1B).

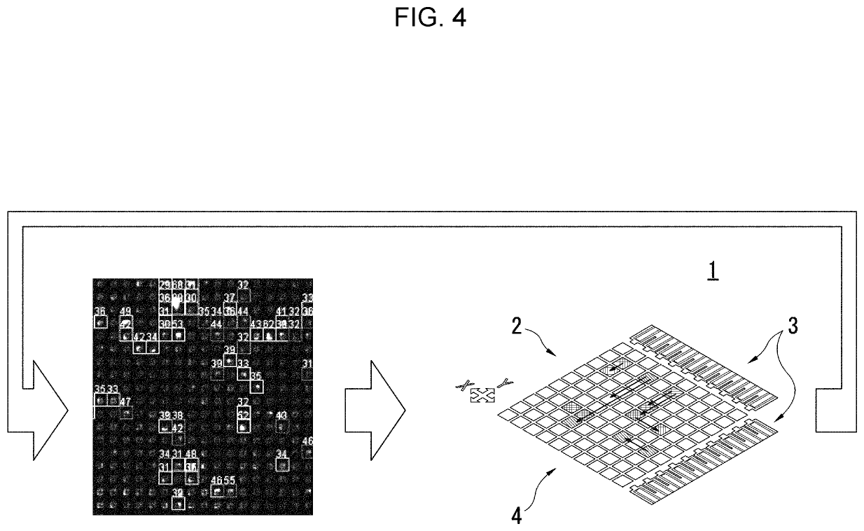

The stage unit 2 is configured to allow a cell incubator (the microwell array 4) to be mounted and move the microwell array 4 in the X-axis direction and the Y-axis direction via a stage driving mechanism (not shown) (see FIGS. 1A and 4).

The stage driving mechanism is configured to be drive-controlled by the stage drive control unit (not shown) so as to align each well 40 in the microwell array 4 with a corresponding one of contact portions 51 and 61 of probes 50 and 60 of the probe array unit 3 (to be described later).

In this case, a cell incubator need not always be adopted as the predetermined substrate, and a sample to be manipulated is not limited to a biological cell or microbe. For example, the predetermined substrate may be a substrate that causes reaction in a plurality of reagents or chemical substances without using any biological samples.

The stage unit 2 need not always be configured to be movable in the X-axis direction and the Y-axis direction of the microwell array 4 via the stage driving mechanism. For example, the stage unit 2 may be fixed, and the probe array unit 3 may be configured to be movable in the X-axis direction and the Y-axis direction relative to the microwell array 4 on the stage unit 2 via a driving mechanism. Both the stage unit 2 and the probe array unit 3 may be configured to be movable in the X-axis direction and the Y-axis direction.

When each well 40 can be aligned with a corresponding one of the contact portions 51 and 61 of the respective probes 50 and 60, an aligning mechanism is not specifically limited. For example, the present invention can adopt not only the form of strictly controlling the moving distance of the stage unit 2 using a stage drive control unit but also position control based on a combination of an imaging mechanism such as a CCD camera and the alignment marks provided on the stage unit 2 and position control based on coordinate information set on an XY plane.

The number of wells of the microwell array 4 is not specifically limited and can be selected as appropriate in accordance with a target sample to be processed or manipulated.

The probe array unit 3 includes a first probe array 5 and a second probe array 6. The first probe array 5 is constituted by a plurality of probes 50 arranged side by side at a predetermined pitch along the Y-axis direction. The second probe array 6 is constituted by a plurality of probes 60 arranged side by side at a predetermined pitch along the Y-axis direction (see FIGS. 1A and 4).

The first probe array 5 and the second probe array 6 are provided on a plane substantially parallel to a plane formed by the microwell array 4 mounted on the stage unit 2 (a plane formed by the X-axis and Y-axis of the stage unit 2).

The first probe array 5 and the second probe array 6 are provided at a height position where each of the probes 50 and 60 driven in the Z-axis direction can come into contact with a target sample such as a cell inoculated in the gel culture medium 41.

The first probe array 5 and the second probe array 6 are arranged substantially in the form of the letter “L” in the same plane (see FIG. 1A).

In this case, the numbers of the plurality of probes 50 and 60 are not specifically limited and can be changed as appropriate depending on the specifics of a target sample to be manipulated. In addition, the numbers of the plurality of probes 50 and 60 may be the same or different from each other.

In addition, the pitch of the plurality of probes 50 and the pitch of the plurality of probes 60 need not always be set to a predetermined pitch. For example, the present invention can adopt a probe array group having a group of probes arranged side by side at different intervals between the probes. Although the pitch of the plurality of probes 50 and the pitch of the plurality of probes 60 are not limited to any specific pitch, the probes are preferably formed at a pitch between the probes in the range of 100 nm or more and 5 mm or less in terms of manipulating a sample such as a cell or microbe.

The probe array unit 3 need not always be constituted by the two probe arrays, that is, the first probe array 50 and the second probe array 60. For example, the present invention can adopt a probe array unit constituted by one probe array or three or more probe arrays. In the form constituted by two or more probe arrays, the placement of each probe array can be designed and changed as appropriate. For example, a plurality of probe arrays may be arranged side by side in accordance with the arraying direction of a plurality of probes. Alternatively, a plurality of probe arrays may be arranged such that a plurality of probes are arrayed in different directions.

The first probe array 50 and the second probe array 60 need not always be arranged substantially in the form of the letter “L” in the same plane. Note, however, that the first probe array 50 and the second probe array 60 are preferably arranged substantially in the form of the letter “L” in the same plane because the first probe array 50 and the second probe array 60 do not interfere with each other while the respective probe arrays are arranged as close to each other as possible, and the moving distance by which the probe array unit and the substrate mounting unit are moved relatively when the probes are made to act at a plurality of desired positions on the substrate.

The probe 50 is constituted by the contact portion 51 that can come into contact with the gel culture medium 41, etc., on the microwell array 4 and is vertically driven along the Z-axis direction and a main body portion 52 that supports the contact portion 51 (see FIG. 1B). Each of the probes 50 is configured to be driven individually.

The main body portion 52 is a member in charge of driving, information processing, etc., associated with drive control on the probe 50 (the functions of a control circuit, communication, power supply, distortion sensor, heat sensor, microheater, etc.).

The surface of the contact portion 51 is formed from a soft actuator material that has flexibility and excellent heat/chemical durability, such as an elastomer such as silicone resin, paper, polymer film, and Teflon□.

The probe 60 has a structure similar to that of the probe 50. That is, the probe 60 is constituted by a contact portion and a main body portion (no reference symbols).

In this case, the material for the contact portion 51 (or the contact portion of the probe 60) is not limited to an elastomer such as silicone resin, paper, polymer film, and Teflon® described above as long as the material has flexibility and excellent heat/chemical durability.

The structures of the contact portion 51 and the main body portion 52 (or the contact portion 61 and the main body portion of the probe 60) are not specifically limited as long as they can be driven along the Z-axis direction and drive-controlled.

The present invention can adopt various types of structures of drive sources for driving the probes 50 and 60 along the Z-axis direction. For example, the present invention can adopt a scheme of driving a flexible actuator by heat driving, electrostatic driving, dielectric elastomer, electromagnetic driving, or light driving or a scheme of mechanically driving the actuator via a wire, etc.

An outline of an operation for contact manipulation with the probe 50 (or the probe 60) will be described with reference to FIG. 1B. Here, FIG. 1B shows, as target samples, different kinds of fungus bodies including fungus body A, fungus body B, and fungus body C in the gel culture media 41 of the microwell array 4. FIG. 1B also shows the reagent gel 42 and the disinfectant gel 43 as target samples.

The contact portion 51 of the probe 50 can be vertically driven along the direction denoted by symbol Z (Z-axis direction) and can come into contact with the gel culture medium 41, etc., at a position where the contact portion 51 is vertically moved downward.

The microwell array 4 can move in the direction denoted by symbol H and can align the position of each well 40 with the position of the contact portion 51. Note that the direction denoted by symbol H is either the X-axis direction or the Y-axis direction. The microwell array 4 can move in the back and forth direction of the drawing surface in FIG. 1B (the direction perpendicular to the direction denoted by reference symbol H).

For example, as indicated by the arrows denoted by reference symbol S1, the contact portion 51 of the probe 50 is vertically driven downward to pick up fungus body A independently cultivated in the individual well 40 and is vertically driven upward. Subsequently, the microwell array 4 is moved in the direction denoted by reference symbol H while fungus body A adheres to the microwell array 4, and the contact portion 51 is vertically driven downward at the position of each co-culturing gel culture medium 41 of a lower layer connection type in which fungus body B or C is cultivated, thereby inoculating the fungus body.

Likewise, as indicated by the arrow denoted by reference symbol S2, fungus body B independently cultivated in the individual well 40 can be inoculated in the co-culturing gel culture medium 41 of the lower layer connection type by driving the contact portion 51 of the probe 50.

A reagent can also be added to the fungus body cultivated in each well 40 or the gel culture medium fed with no fungus body by making the contact portion of the probe 50 in contact with the reagent gel 42 come into contact with the fungus body or the gel culture medium.

In addition, bringing the contact portion 51 of each probe 50 fed with a fungus body into contact with the disinfectant gel 43 can perform contact manipulation for desterilizing the fungus body adhering to the contact portion 51. Furthermore, although not shown, it is possible to separately provide a cleaning gel including a cleaning liquid for cleaning the contact portion 51 in contact with the disinfectant gel 43. Desterilizing and cleaning the contact portion 51 makes it possible to perform contact manipulation again with respect to a fungus body.

More specifically, the manipulation apparatus 1 can execute the above manipulation in combination with the following basic manipulation. Although the following description is made with reference to a fungus body (microbe) as a target sample, it is possible to perform manipulation using other types of cells.

### (1) Subculture Culturing/Re-culturing

Manipulation of picking up a fungus body from a given well (or a plate, laboratory dish, etc.) in which the fungus body is cultivated and inoculating the fungus body into a gel culture medium inoculated with no fungus body.

### (2) Filling

Manipulation of inoculating fungus bodies from a well (or a given plate, laboratory dish, etc.) in which fungus bodies are initially inoculated randomly and proliferated sparsely into a gel culture medium inoculated with no fungus body in another well.

### (3) Reagent Addition

Manipulation of picking up a small number of reagents from a well filled with a reagent gel and adding the reagent to a fungus body in another well or a gel culture medium inoculated with no fungus body by bringing the reagent into contact with the fungus body or the gel culture medium.

### (4) Screening

Manipulation of picking up a fungus body from a well (or a given plate, laboratory dish, etc.) specified in various types of analysis steps (proliferation analysis, metabolism analysis, image analysis using fluorescence, etc.) and inoculating the fungus body in a gel culture medium inoculated with no fungus body in another well.

### (5) Co-Culturing

Manipulation of picking up fungus bodies from a plurality of specified wells (or plates, laboratory dishes, etc.) and inoculating a plurality of fungus bodies in a gel culture medium inoculated with no fungus body in another well.

### (6) Probe Cleaning

Manipulation of regenerating the contact portion of a probe used for contact manipulation with respect to a fungus body or reagent movement by, for example, bringing the contact portion of the probe into contact with a disinfectant gel and a cleaning gel and drying the contact portion of the probe.

In this case, a well filled with a reagent, that is, the reagent, need not always be formed into a gel, and the present invention can adopt a form in which a well is filled with a liquid reagent.

As described above, the manipulation apparatus 1 can efficiently perform various contact manipulations for various types of uses of fungus bodies, cells, etc., by using the plurality of probes 50 and the plurality of probes 60.

The manipulation apparatus 1 to which the present invention is applied can further include the following mechanism.

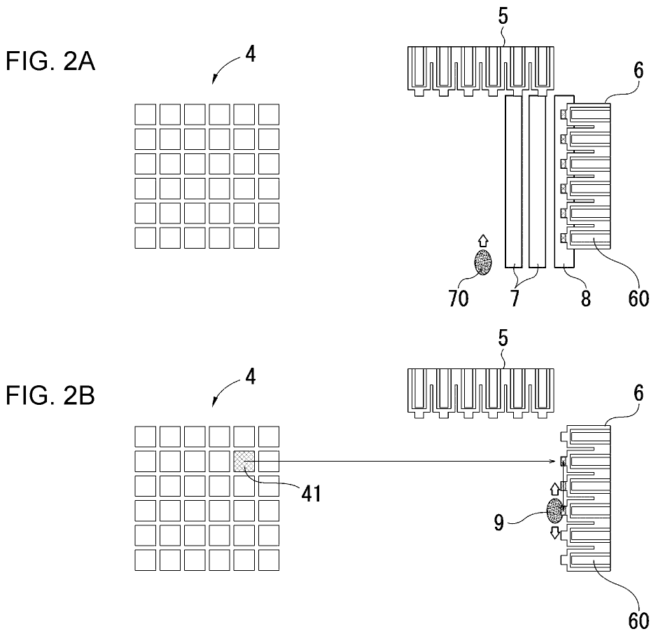

As shown in FIG. 2A, the microwell array 4 can be provided with a cleaning tank 7 and a sterilizing tank 8 formed along the arraying direction of the plurality of probes 60 of the second probe array 6. Note that the cleaning tank 7 and the sterilizing tank 8 in this case correspond to probe processing units in the claims of the present application.

The cleaning tank 7 is filled with a cleaning liquid for cleaning the contact portion of the probe 60. The sterilizing tank 8 includes a mechanism for sterilizing the contact portion of the probe 60 by UV or heating. The cleaning tank 7 and the sterilizing tank 8 can collectively clean or sterilize the contact portions of the plurality of probes 60 constituting the second probe array 6 by being brought into contact with the contact portions. In addition, a structure similar to the cleaning tank 7, etc., can be filled with a reagent to collectively make a reagent adhere to the respective probes.

As shown in FIG. 2A, in place of the cleaning tank 7, a mechanical cleaning unit 70 can be provided, which comes into contact with the contact portion of the probe 60 and physically removes a fungus body or reagent, etc., adhering to the contact portion. As the mechanical cleaning unit 70, for example, a brush can be used, which rubs the contact portion of the probe 60 and rotates while the contact portion is caught in the brush. The mechanical cleaning unit 70 is configured to move along the arraying direction of the plurality of probes 60 via guide rails, etc. (not shown). Note that the mechanical cleaning unit 70 in this case corresponds to a probe contact portion in the claims of the present application.

In this case, referring to FIG. 2A, the cleaning tank 7 and the sterilizing tank 8 are provided only on the second probe array 6 side. However, the first probe array 5 side can also be provided with a cleaning tank and a sterilizing tank that collectively clean or sterilize the contact portions 51 of the plurality of probes 50 constituting the first probe array 5 by bringing the contact portions 51 into contact with the tanks.

In addition, referring to FIG. 2A, the mechanical cleaning unit 70 is provided only on the second probe array 6 side. However, likewise, the first probe array 5 side can also be provided with a mechanical cleaning unit that can move along the arraying direction of the plurality of probes 50 and physically removes a fungus body, reagent, etc., adhering to the contact portions 51.

Thus, the manipulation apparatus 1 can more efficiently perform manipulation accompanying contact with a fungus body or cell by providing the manipulation apparatus 1 with a mechanism that can collectively clean and sterilize one probe array or make a reagent adhere to the probe array.

As a sterilizing mechanism in the manipulation apparatus to which the present invention is applied, for example, a mechanism for sterilizing each probe by locally irradiating it with a UV laser can be adopted other than the mechanisms described above.

As shown in FIG. 2B, this apparatus can also be provided with a coating mechanism 9 that comes into contact with one contact portion of the probe 60 which has taken in a fungus body from the gel culture medium 41 in which a specific fungus body has been cultivated and coats another contact portion with the fungus body, reagent, etc., taken in by the contact portion. As the coating mechanism 9, for example, a brush can be adopted, which rubs the contact portion of the probe 60 and rotates while the contact portion is caught in the brush. The coating mechanism 9 is configured to move along the arraying direction of the plurality of probes 60 via guide rails, etc. (not shown). Note that the coating mechanism 9 in this case corresponds to a probe contact portion in the claims of the present application.

In this case, referring to FIG. 2B, the coating mechanism 9 is provided only on the second probe array 6 side. However, likewise, the first probe array 5 side can also be provided with a coating mechanism that can move along the arraying direction of the plurality of probes 50 and applies the fungus body, reagent, etc., adhering to one contact portion 51 onto another contact portion 51.

Thus, the manipulation apparatus 1 can more efficiently perform manipulation accompanying contact with a fungus body or cell by providing the manipulation apparatus 1 with a mechanism that collectively coats one probe array with a fungus body or reagent.

The manipulation apparatus 1 can be an apparatus combined with an imaging mechanism such as a CCD camera. This makes it possible to perform position control based on the information on a captured image, and hence can further improve the accuracy associated with alignment between the stage unit 2 (microwell array 4) and the probe array unit 3.

Combining the manipulation apparatus 1 with an imaging mechanism such as a CCD camera allows observation of a culturing state, etc. That is, the manipulation apparatus 1 enables efficient operations in accordance with purposes by, for example, determining a cell that can be identified by determining the presence/absence and shape of the cell itself and by irradiation with light having a specific wavelength as well as performing alignment between the stage unit 2 and the probe array unit 3.

The manipulation apparatus 1 may be used in combination with a culturing tank having a temperature adjusting function and a sterilizing function such as a UV lamp. This form makes it possible to perform processing accompanying contact manipulation with respect to a target sample under an environment including culturing conditions for a fungus body or cell. For example, the manipulation apparatus 1 can be placed inside a culturing tank.

The details of the manipulation apparatus 1 described above are merely exemplary, and the manipulation apparatus 1 can be used in combination with other types of culturing devices or analysis devices.

An example of specific manipulation using the manipulation apparatus 1 will be further described below.

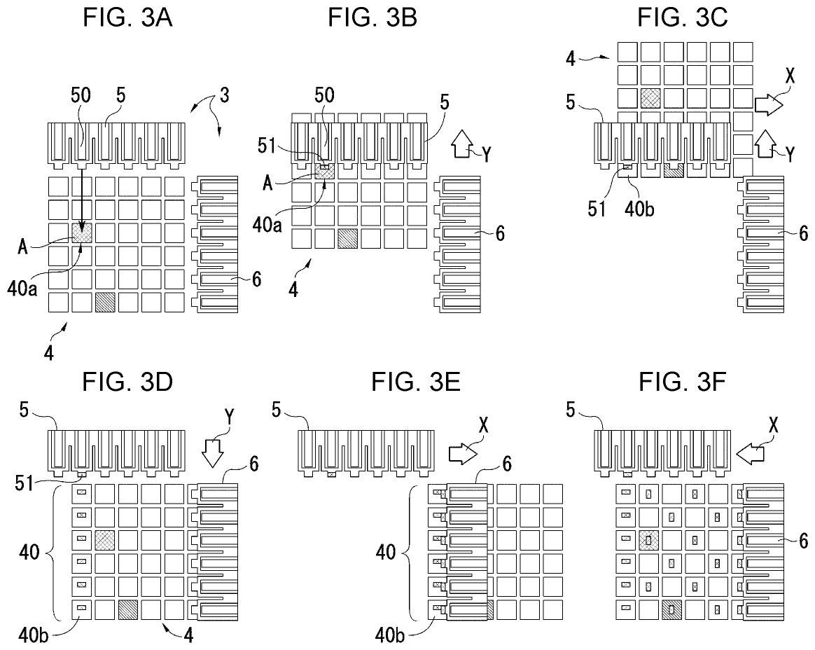

FIGS. 3A-F show a sequence of seeding a cell sample cultivated in a specific well of the microwell array 4 into a plurality of wells of the same microwell array 4 in an arbitrary pattern by using the manipulation apparatus 1.

First, FIG. 3A shows the state of the initial placement of the probe array unit 3 and the microwell array 4. In a well 40a of the microwell array 4, specific cell sample A to be seeded in another well is cultivated.

The microwell array 4 (stage unit 2) in this initial placement state is moved upward in the Y-axis direction (the direction denoted by reference symbol Y in FIG. 3B), and the contact portion 51 of the second probe 50 from the left in FIG. 3B is aligned with the well 40a. At the same position, the probe 50 is moved up and down along the Z-axis direction to bring the contact portion 51 into contact with the well 40a, thereby picking up cell sample A (see FIG. 3B).

Subsequently, the microwell array 4 is moved to the right in the X-axis direction and up in the Y-axis direction (the directions denoted by reference symbols X and Y in FIG. 3C) to align the contact portion 51 that has picked up cell sample A with a leftmost lower well 40b of the microwell array 4 (see FIG. 3C).

The microwell array 4 is then moved downward in the Y-axis direction (the direction denoted by reference symbol Y in FIG. 3D) to align the contact portion 51 that has picked up cell sample A with each well 40 in the leftmost column of the microwell array 4 along the Y-axis direction, which includes the well 40b. The microwell array 4 is stopped at each well 40, and the probe 50 is moved up and down along the Z-axis direction to bring the contact portion 51 into contact with the well 40, thereby seeding cell sample A (see FIG. 3D).

Next, the microwell array 4 is moved to the right in the X-axis direction (the direction denoted by reference symbol X in FIG. 3E) to align each well 40 in the leftmost column in which cell sample A is seeded with the contact portions 61 of the plurality of probes 60 of the second probe array 6 (see FIG. 3E). At the same position, the probe 60 is moved up and down along the Z-axis direction to bring the contact portion 61 into contact with each well 40 to pick up cell sample A (see FIG. 3E). This sets a state in which cell sample A collectively adheres to the plurality of probes 60.

In addition, the microwell array 4 is moved to the left in the X-axis direction (the direction denoted by reference symbol X in FIG. 3F) to seed cell sample A picked up by the plurality of probes 60 at the position of an arbitrarily set well (see FIG. 3F).

In the example shown in FIG. 3F, the first, third, and fifth probes 60, counted from above, of the plurality of probes 60 on the second and fourth columns from the left side of the microwell array 4 in FIG. 3F are moved up and down along the Z-axis direction to bring the contact portion 61 into contact with each well 40 to seed cell sample A. In addition, the second, fourth, and sixth probes 60, counted from above, of the plurality of probes 60 on the third and fifth columns from the left side of the microwell array 4 in FIG. 3F are moved up and down along the Z-axis direction to bring the contact portion 61 into contact with each well 40 to seed cell sample A. As a result, cell sample A is seeded in the respective wells of the microwell array 4, except for the leftmost column, in a staggered pattern (see FIG. 3F).

The seeding pattern of cell sample A shown in FIGS. 3A-F can be arbitrarily set, and is not necessarily limited to the form in which cell sample A is seeded in a staggered pattern. In addition, the number of types of cell samples to be seeded is not limited to one, and a plurality of types of cell samples can be handled.

Thus, cell samples can be efficiently seeded in a large number of wells of the microwell array 4 by using the manipulation apparatus 1. The manipulation apparatus 1 can considerably improve the efficiency of a series of operations as compared with, for example, the manipulation using a colony picking apparatus that brings a conventional needle having a minute distal end into contact with a cell sample.

FIG. 4 schematically shows how analysis manipulation for a target sample and culturing manipulation are repeatedly performed by using the manipulation apparatus 1. As shown in FIG. 4, for example, a microbe sample is cultivated in the microwell array 4, and image analysis is performed based on the development of a fluorescence protein (see the left view of FIG. 4). A specific microbe is then inoculated in a well inoculated with no fungus body in the microwell array 4 after the analysis, or a plurality of fungus bodies are inoculated and co-cultivated in the same well (see the right view of FIG. 4).

In the microwell array 4 after culturing, analysis manipulation such as image analysis can be performed again by making the probe array unit 3 bring a reagent into contact with the cultivated microbe sample. Note that the arrows denoted by reference symbols X and Y in FIG. 4 indicate the directions in which the microwell array 4 moves.

Thus, analysis manipulation and culturing manipulation can be repeatedly performed in the same microwell array 4, and experiment manipulation can be performed without the trouble of moving a target sample to a separate substrate (a microwell array, laboratory dish, etc.).

Referring to FIG. 4, image analysis using fluorescence is exemplified as analysis manipulation. However, analysis manipulation is not limited to this. For example, it is possible to perform analysis on the proliferation rate of a target sample and metabolism analysis on the amount of metabolite, metabolism rate, etc.

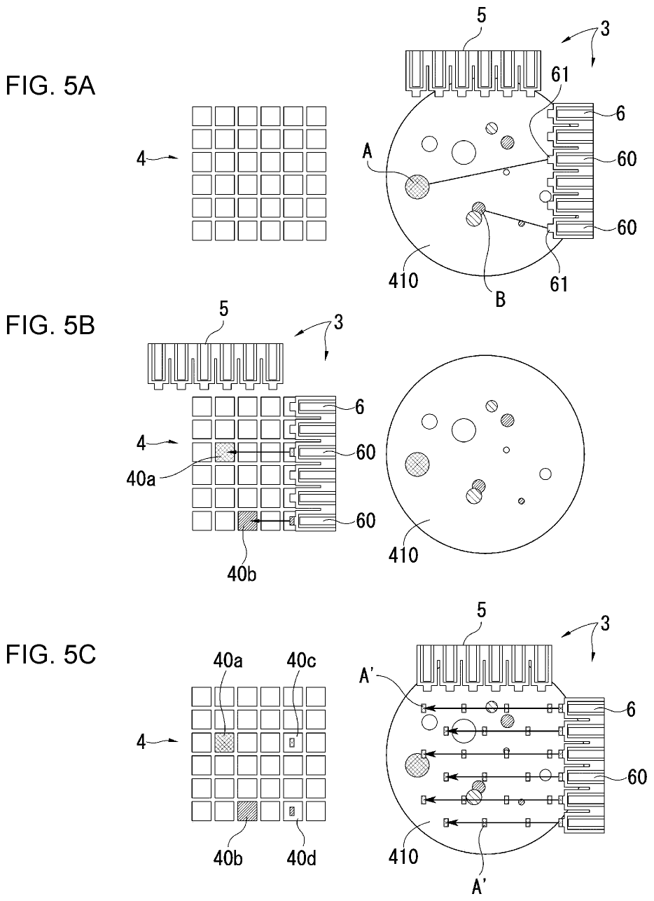

FIGS. 5A-C schematically show manipulation of inoculating a target sample between a gel plate 410 formed on a laboratory dish and the microwell array 4 by using the manipulation apparatus 1.

As shown in FIG. 5A, a plurality of microbe colonies (or cell tissue pieces) are cultivated on the gel plate 410. The gel plate 410 is placed on a stage unit (not shown), and the probe array unit 3 picks up a specific sample.

For example, the gel plate 410 is moved in the X-axis direction or Y-axis direction to pick up colony A and colony B on the gel plate 410 with the contact portions 61 of the two probes of the second probe array 6.

Subsequently, on the stage unit, the gel plate 410 is moved onto the microwell array 4, and colony A and colony B picked up with the contact portions 61 of the two probes 60 are inoculated in the well 40a or 40b (see FIG. 5B).

In addition, as shown in FIG. 5C, on the microwell array 4, colony A and colony B can be inoculated in a well 40c or 40d by using the probe array unit 3.

Furthermore, as shown in FIG. 5C, colony A can be inoculated at a desired position on the gel plate 410 again by coating the contact portions 61 of all the probes 6 on the second probe array 6 with colony A.

Thus, using the manipulation apparatus 1 makes it possible to perform manipulation of inoculating a target sample between the gel plate 410 formed on a laboratory dish and the microwell array 4.

The manipulation apparatus 1 to which the present invention is applied can add a reagent to a spot as a manipulation target at an arbitrary timing by contact manipulation with a plurality of probes. For example, when a plurality of compounds (reagents, etc.) are mixed and added at a spot as a manipulation target, an adding operation can be easily performed upon adjustment of the blending ratios of the compounds.

In this case, when adjusting the blending ratios, the present invention may adopt a form of performing adjustment based on the number of times of probe contact or a form of separately providing an injection mechanism for a reagent, etc., in each probe and adjusting the amount of reagent to be added. As described above, the manipulation apparatus 1 according to the present invention can efficiently mix a plurality of targets such as compounds.

In addition, for example, dispersing candidate group A and candidate group B onto a microwell array of 100 rows×100 columns and executing combination experiment (for example, dispersing different cell samples for each column and different reagents for each row) makes it possible to also perform manipulation of efficiently searching out a combination exhibiting the highest reaction level.

### Second Embodiment

Next, a manipulation apparatus 1A as an example of a manipulation apparatus to which the present invention is applied will be described. In describing the manipulation apparatus 1A, a detailed description of the structures and functions of members that overlap those in the above-described first embodiment of the present invention will be omitted.

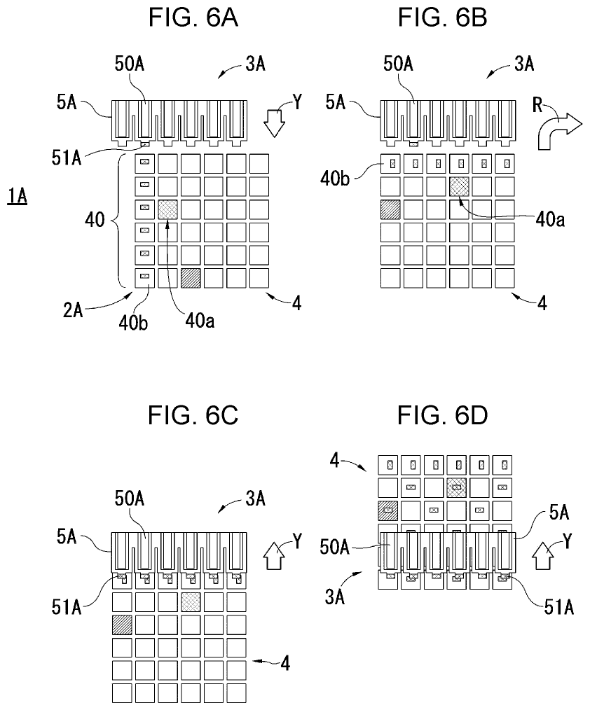

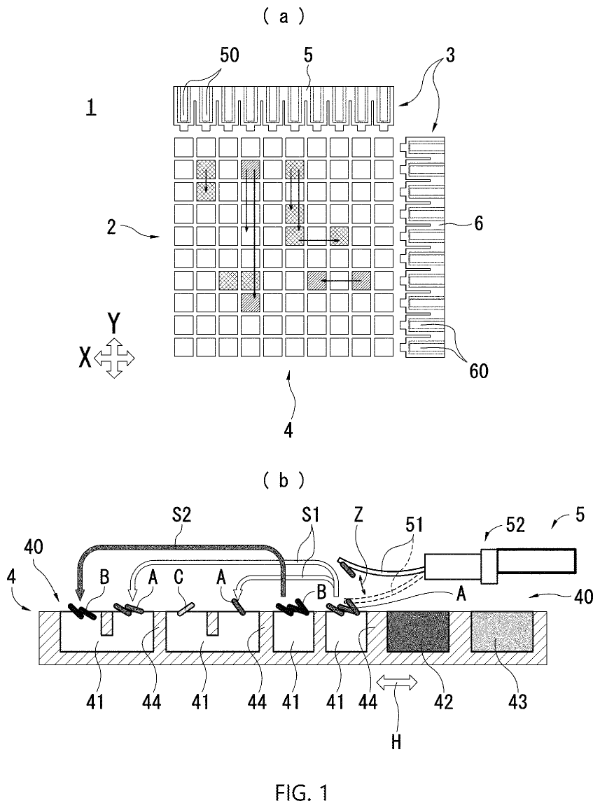

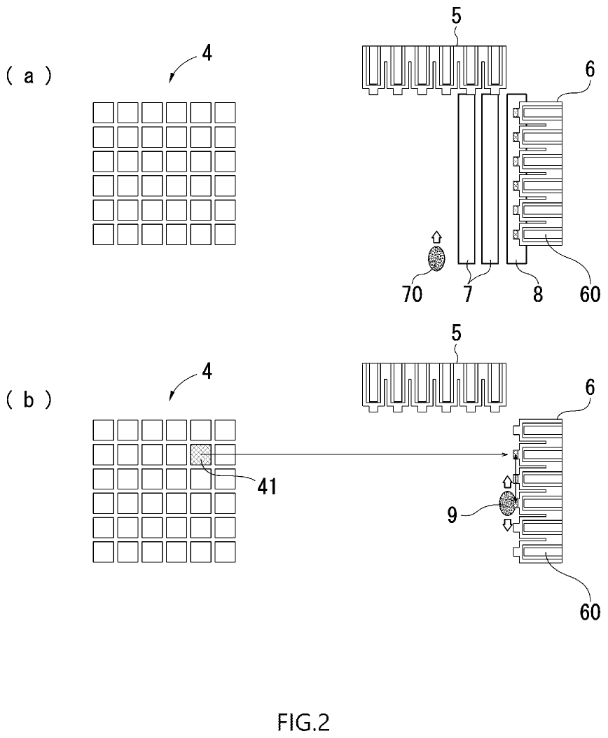

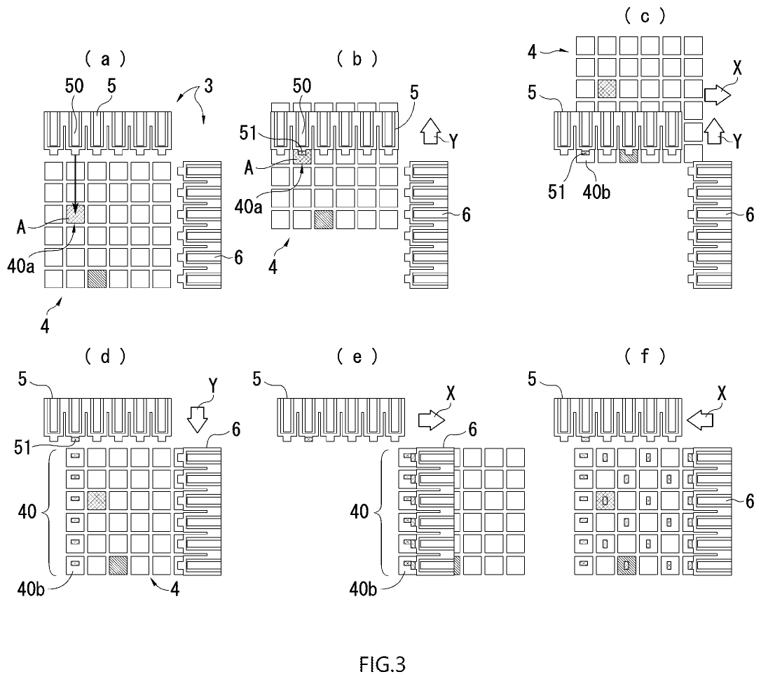

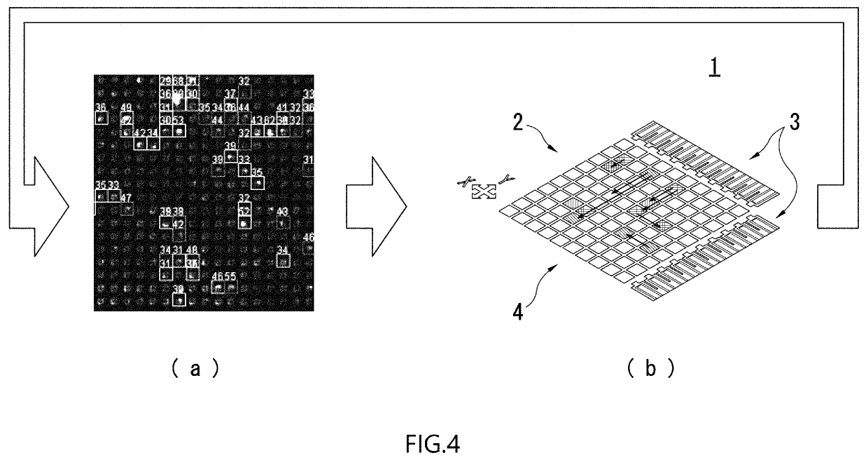

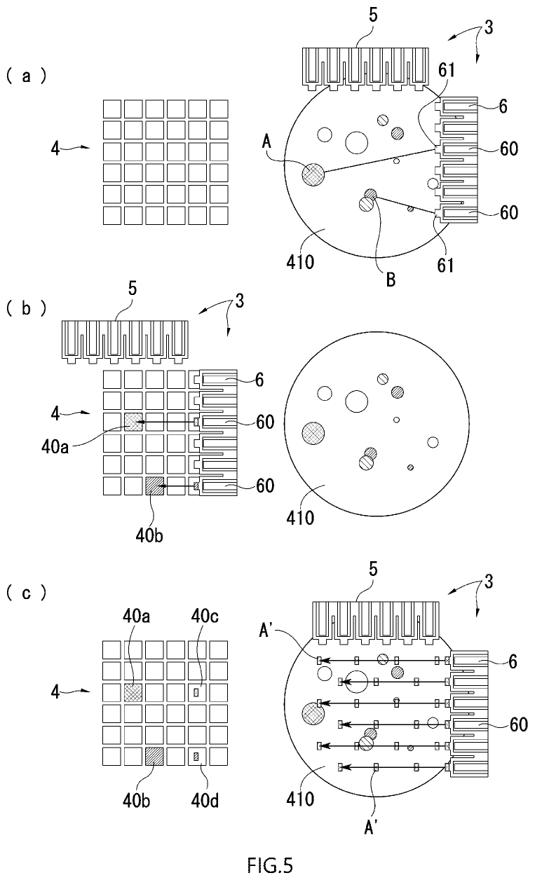

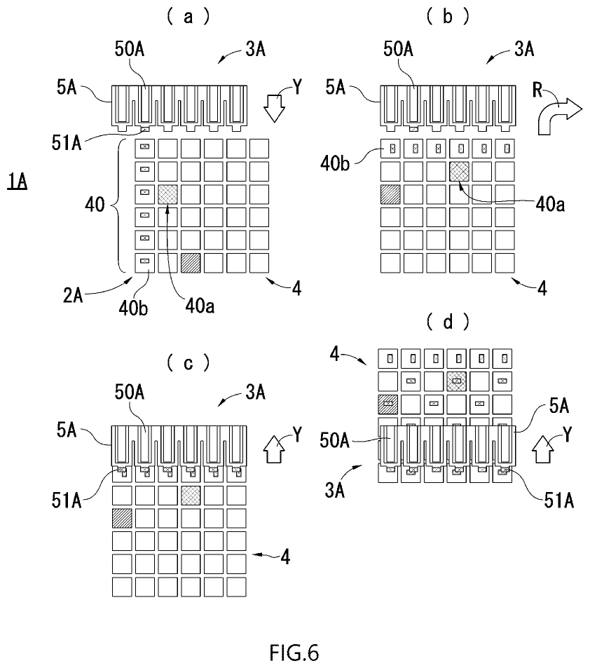

The manipulation apparatus 1A shown in FIG. 6A includes a stage unit 2A on which a predetermined cell incubator is mounted and a probe array unit 3A including a plurality of probes 50A. In this case, a microwell array 4 is exemplified as a cell incubator.

The cell incubator (the microwell array 4) can be mounted on the stage unit 2A, and the microwell array 4 can be moved in the X-axis direction and the Y-axis direction via a stage driving mechanism (not shown) (see FIG. 6A). The stage unit 2A is configured to make the microwell array 4 pivot about the Z-axis perpendicular to both the X-axis and the Y-axis via the stage driving mechanism.

The stage driving mechanism is configured to be drive-controlled by the stage drive control unit (not shown) so as to align each well 40 in the microwell array 4 with a contact portion 51A of a corresponding one of the probes 50A of the probe array unit 3A (to be described later).

The manipulation apparatus 1A according to the second embodiment differs from the manipulation apparatus 1 according to the first embodiment described above in that the number of probe arrays is one (one axis), and the stage unit 2A is configured to be pivotal about the Z-axis.

In this case, the stage unit 2A need not always be configured to move the microwell array 4 in the X-axis direction and the Y-axis direction and be pivotal about the Z-axis via the stage driving mechanism. For example, the stage unit 2A may be fixed and the probe array unit 3A may be configured to be movable in the X-axis direction and the Y-axis direction and pivotal about the Z-axis with respect to the microwell array 4 on the stage unit 2A via the driving mechanism. In addition, both the stage unit 2A and the probe array unit 3A may be configured to be movable in the X-axis direction and the Y-axis direction and pivotal about the Z-axis.

The probe array unit 3 includes a probe array 5A. The probe array 5A is constituted by a plurality of probes 50A arranged side by side at a predetermined pitch along the X-axis direction (see FIG. 6A). The probe array 5A is provided on a plane substantially parallel to a plane defined by the microwell array 4 mounted on the stage unit 2A (a plane defined by the X-axis and the Y-axis of the stage unit 2A).

The probe array 5A is provided at a height position at which each probe 50A to be driven can come into contact with a target sample such as a cell inoculated in a gel culture medium 41 in the Z-axis direction. The probe 50A is constituted by the contact portion 51A (see FIG. 6A) and a main body portion (not shown). The probe array 5A has a structure similar to that of the first probe array 5 described above.

An example of specific manipulation using the manipulation apparatus 1A will be described below.

FIG. 6 shows a sequence of seeding a cell sample cultivated in a specific well of the microwell array 4 into a plurality of wells of the same microwell array 4 in an arbitrary pattern by using the manipulation apparatus 1A. Note that the sequence of manipulation using the manipulation apparatus 1A (probe array 5A) up to FIG. 6A is similar to the manipulation performed by the manipulation apparatus 1 (first probe array 5) shown in FIGS. 3A-D, and hence a description of the sequence will be omitted.

As shown in FIG. 6A, cell sample A cultivated in a well 40a is picked up by the contact portion 51A of the probe 50A and seeded in each well 40 in the leftmost column of the microwell array 4 along the Y-axis direction, which includes a well 40b. Note that the arrow denoted by reference symbol Y shown in FIG. 6A indicates the direction in which the microwell array 4 (stage unit 2A) moves in the Y-axis direction.

Next, the microwell array 4 is made to pivot clockwise about the Z-axis (the direction denoted by reference symbol R in FIG. 6B) to align each well 40 in the uppermost column in which cell sample A is seeded with a corresponding one of the contact portions 51A of the plurality of probes 50A of the probe array 5A. In addition, at the same position, the probes 50A is moved up and down along the Z-axis direction to bring the contact portion 51A into contact with each well 40 to pick up cell sample A (see FIG. 6C). This sets a state in which cell sample A collectively adheres to the plurality of probes 50A.

Thus, the microwell array 4 is moved upward in the Y-axis direction (the direction denoted by reference symbol Y in FIGS. 6C and 6D) to seed cell sample A picked up by the plurality of probes 50A at the position of an arbitrarily set well (see FIG. 6D).

Thus, cell samples can also be efficiently seeded in a large number of wells of the microwell array 4 by using the manipulation apparatus 1A. Manipulation using the manipulation apparatus 1A can significantly improve the efficiency of a series of operations as compared with, for example, a conventional colony picking apparatus that brings a needle having a minute distal end into contact with a sample.

The manipulation apparatus to which the present invention is applied can perform manipulations accompanying contact with a small number of samples, such as inoculating, sample splitting, selective co-culturing, and reagent addition, with respect to individual culturing samples on the order of several tens of thousands to several hundreds of thousands in parallel on a large scale.

This makes it possible to construct a large-scale parallel culturing analysis experiment system that can execute analysis and culturing on the order of several hundreds of thousands by combining this apparatus with a microarray type analysis system that can acquire an enormous amount of information.

In addition, it is possible to dramatically improve the operation efficiency of manipulation accompanying contact with a sample with respect to a cell sample or microbe sample cultivated on a plate or a laboratory dish on which a cell piece is placed as well as combining the apparatus with a microarray type analysis system.

Furthermore, the manipulation apparatus to which the present invention is applied can perform proper manipulation with respect to a small number of samples, and hence allows reduction in the amount of valuable reagent or the amount of medium. It is, therefore, expected to reduce the operational cost for experiment manipulation.

As described above, the manipulation apparatus according to the present invention can efficiently perform culturing, manipulation, and analysis of minute samples such as cells and microbes on a large scale and can perform experiment manipulation using a small number of reagents, etc.

The manipulation method according to the present invention can efficiently perform culturing, manipulation, and analysis of minute samples such as cells and microbes on a large scale and can perform experiment manipulation using a small number of reagents, etc.

While the present invention made by the present inventor has been concretely described above based on the embodiments, the present invention is not limited to the above-described embodiments since various changes may be made within the scope of the present invention without deviating from the spirit thereof.

### LIST OF REFERENCE CHARACTERS

1: Manipulation apparatus

2: Stage unit

3: Probe array unit

4: Microwell array

40: Well

41: Gel culture medium

42: Reagent gel

43: Disinfectant gel

44: Partition wall

5: First probe array

50: Probe

51: Contact portion

52: Main body portion

6: Second probe array

60: Probe

61: Contact portion

7: Cleaning tank

70: Mechanical cleaning unit

8: Sterilizing tank

9: Coating mechanism

1. A manipulation apparatus comprising:
a substrate mounting unit on which a predetermined substrate is mounted; and
a probe array unit configured to drive as to contact a target sample on the predetermined substrate,
wherein the probe array unit include a plurality of probes arranged side by side,
wherein the probe array unit is provided to face the substrate mounting unit, and
wherein the probe array unit is movable relative to the substrate mounting unit.

2. The manipulation apparatus according to claim 1, wherein the probe array unit further includes:
at least a first probe array which includes a first set of probes from the plurality of probes arranged side by side in a first direction; and
a second probe array which includes a second set of probes from the plurality of probes arranged side by side in a second direction,
wherein the second direction is different from the first direction.

3. The manipulation apparatus according to claim 2, wherein the probe array unit is configured to be movable back and forth relative to the substrate mounting unit in an X-axis direction and a Y-axis direction, the Y-axis direction being perpendicular to the X-axis direction,
wherein the first direction is the X-axis direction or the Y-axis direction, and
wherein the second direction is one of the X-axis direction and the Y-axis direction, and the second direction is perpendicular to an arraying direction of the first probe array.

4. The manipulation apparatus according to claim 1, wherein the probe array unit is configured to move back and forth relative to the substrate mounting unit in an X-axis direction and a Y-axis direction, the Y-axis direction being perpendicular to the X-axis, and
wherein the probe array unit is further configured to pivot relative to the substrate mounting unit in a θ direction around a Z-axis, the Z-axis being perpendicular to the X-axis direction and the Y-axis being perpendicular to the X-axis, and
wherein the plurality of probes are arranged side by side in one of the X-axis direction and the Y-axis direction.

5. The manipulation apparatus according to claim 3, wherein the first probe array and the second probe array are arranged substantially in a form of a letter “L.”

6. The manipulation apparatus according to claim 1, further comprising a probe processing unit that is formed substantially parallel to an arraying direction of the plurality of probes at a position and at a length that allow the plurality of probes to come into contact with the probe processing unit, and
wherein the probe processing unit is configured to clean or sterilize the probes, or make a reagent adhere to the probes.

7. The manipulation apparatus according to claim 1, further comprising a probe contact portion configured to move substantially parallel to the arraying direction of the probes and come into contact with the plurality of probes to clean or sterilize at least one of the probes, or make a target sample adhere to at least one of the probes.

8. The manipulation apparatus according to claim 1, wherein the plurality of probes are formed at a pitch in a range of not less than 100 nm and not more than 5 mm.

9. The manipulation apparatus according to claim 1, wherein the plurality of probes are vertical probes that are configured to be individually controllable, with a distal end of each of the vertical probes moving close to or away from the predetermined substrate.

10. The manipulation apparatus according to claim 1, wherein the predetermined substrate is a microwell array on which not less than 100 wells per cm² are formed.

11. A manipulation method comprising:
a moving step of moving, relative to a substrate mounting unit on which a predetermined substrate is mounted, a probe array unit having a plurality of probes arranged side by side to face the substrate mounting unit; and
a contact step of driving the probe array unit to a desired position to bring at least one of the probes into contact with a target sample on the predetermined substrate.

12. The manipulation method according to claim 11, wherein the probe array unit includes:
at least a first probe array which includes a first set of the plurality of probes arranged side by side in a first direction; and
a second probe array which includes a second set of the plurality of probes arranged side by side in a second direction,
wherein the second direction is different from the first direction.

13. The manipulation method according to claim 12, wherein the first direction is the X-axis direction or the Y-axis direction,
wherein the second direction is one of the X-axis direction and the Y-axis direction and is a direction perpendicular to an arraying direction of the first probe array, and
wherein, in the moving step, the probe array unit is moved back and forth relative to the substrate mounting unit in the X-axis direction and the Y-axis direction.

14. The manipulation method according to claim 10, wherein the probe array unit is provided with a probe array on which the plurality of probes are arranged side by side in an X-axis direction or a Y-axis direction, the Y-axis direction being perpendicular to the X-axis, and
wherein, in the moving step, the probe array unit is moved back and forth relative to the substrate mounting unit in the X-axis direction and the Y-axis direction, and the probe array unit is made to pivot relative to the substrate mounting unit in a θ direction around a Z-axis, the Z-axis being perpendicular to the X-axis and the Y-axis.

15. The manipulation method according to claim 11, further comprising:
a culturing step of culturing a target sample on the predetermined substrate; and
an analysis step of performing image analysis with respect to the target sample cultivated on the predetermined substrate,
wherein the culturing step and the analysis step are repeatedly performed with respect to the predetermined substrate.
